(19) United States
(12) Patent Application Publication
(10) Pub. No.: US 2023/0220493 A1
(43) Pub. Date: Jul. 13, 2023

(54) Diagnosis of Antibiotic Resistance Genes in Clinical Specimens

(71) Applicant: AOS Technolgies LLC, Ridgeland, MS (US)

(72) Inventors: Anshul Anugu, Ridgeland, MS (US); Shade Smith, Moorhead, MS (US); Alex Oliveri, Mandeville, LA (US)

(73) Assignee: AOS Technologies LLC, Ridgeland, MS (US)

(21) Appl. No.: 17/571,774

(22) Filed: Jan. 10, 2022

(51) Int. Cl. C12Q 1/689; C12Q 1/686; C12Q 1/6837; G01N 21/64

(52) U.S. Cl. CPC C12Q 1/689; C12Q 1/686; C12Q 1/6837; G01N 21/6428; G01N 2021/6439

(57) ABSTRACT

The invention includes, at minimum, an integrated, automated system for diagnosis of antibiotic resistance genes that performs microarray hybridization, washes, and reading, using a plurality of high-density glass and/or polymer microarray chips. The system may include one or more ring or circle-shaped microarray chips formed from glass and/or a polymer. The system may also include well plates pre-treated with one or more of fluorescently labeled primers, polymerase, dNTPs or other chemicals or reagents used to multiplex PCR identification of antibiotic resistance genes. The system may also include one or more of 96 or 128 sized well plates. The system may also include one or more centrifugation tubes having a nanopore filter fixed in place and/or a viscous solution for isolating smaller pathogens from larger host eukaryotic cells.

### FIELD

The present invention relates generally to biomolecules, devices, and procedures for diagnosis of clinical bacterial infections, and in a particular though non-limiting embodiment to a plurality of methods and means for genotypically diagnosing antibiotic resistance mechanisms.

### BACKGROUND

A problem associated with antibiotic resistance is a major concern to global healthcare. In particular, there is a large and rapid emergence of resistant bacteria occurring worldwide, threatening the efficacy of current antibiotics. The emergence of this antibiotic resistance crisis is frequently traced to misuse and overuse of antibiotics. Due to our current health care conditions, and especially the COVID-19 pandemic, this problem has already created a substantial burden on hospitals, health care systems, insurance companies, families, and governments. The CDC classifies the problem as an “urgent issue” and one of the biggest public health challenges of our time. Each year, for example, at least 2.8 million people in the United States contracted an antibiotic resistant infection, which accounted for more than 35,000 deaths. The global antibiotic resistance problem is a massive addressable market, and the need for an efficient and rapid diagnostic test is longstanding.

A primary cause of antibiotic resistance is poor antibiotic stewardship. Broad-spectrum antibiotics are frequently prescribed during initial clinic visits because patients want to leave with a remedy for their illness and the causative agent is not always easily identifiable. Current methods often take too long or cost too much to be used for patients without severe infections. The problem arises because treating such patients with antibiotics results in more resistance. The dilemma, then, is that patients with less severe infections might want antibiotic treatment but treating them is not worth the risk of more drug resistance. A better diagnostic method could alleviate this pressure by reducing the risk of contributing to antibiotic resistance, thus making it worthwhile to treat these patients.

In more severe cases, for example, in an immunosuppressed patient, diagnostic methods are already known. The primary method used by clinics to determine antibiotic resistance is culturing on Kirby-Bauer Disk Diffusion plates. However, this method has drawbacks. One drawback is time. For example, it takes up to two days to plate and isolate bacteria, and then another several hours to determine resistance via culture with antimicrobial agents. During this time, patients are often already prescribed an antibiotic, which contributes to resistance and makes it less effective for successive patients. A second drawback is scope. Many species of pathogen will not grow on agar and cannot be tested. Furthermore, there are far more commercially available antibiotics than can be practically tested with Kirby-Baur.

Antibiotic resistance would not be such a problem if new and effective antibiotics were being frequently discovered. Unfortunately, this is not the case. The pipeline for development of novel antibiotics has stagnated with many new drugs consisting of only slight variations of older drugs. So, clinicians must make better use of the antibiotics currently in circulation by avoiding contributing to further resistance as much as possible. When new drugs are developed, they should be preserved for multi-drug resistant infections that do not respond to current antibiotic therapy. To enable more specialized use of antibiotics, clinicians must have access to a better diagnostic procedure for characterization of antibiotic resistance in an infection.

A better diagnostic method is therefore necessary to combat antibiotic resistance and improve patient outcomes. This diagnostic method must be sufficiently rapid, inexpensive, and must accurately assess resistance of a broad scope of pathogens to most available antibiotics.

### SUMMARY

The present invention includes, at least, an integrated automated system for diagnosis of antibiotic resistance genes, and performs microarray hybridization, washes, and reading, using a plurality of high-density glass and/or polymer microarray chips. In other aspects, the system includes one or more ring or circle-shaped microarray chips formed from glass and/or a polymer. In other aspects, the system includes well plates pre-treated with one or more of fluorescently labeled primers, polymerase, dNTPs or other chemicals or reagents used to multiplex PCR identification of antibiotic resistance genes. In one particular aspect, the system includes one or more of 96 or 128 sized well plates. In still other aspects, the system includes one or more centrifugation tubes having a nanopore filter fixed in place and/or a viscous solution for isolating smaller pathogens from larger host eukaryotic cells.

### BRIEF DESCRIPTION OF THE DRAWINGS

FIG. 1 depicts a ring microarray system according to a first aspect of the invention. is a plan view for automation of a diagnostic test using a ring microarray system according to a second aspect of the invention.

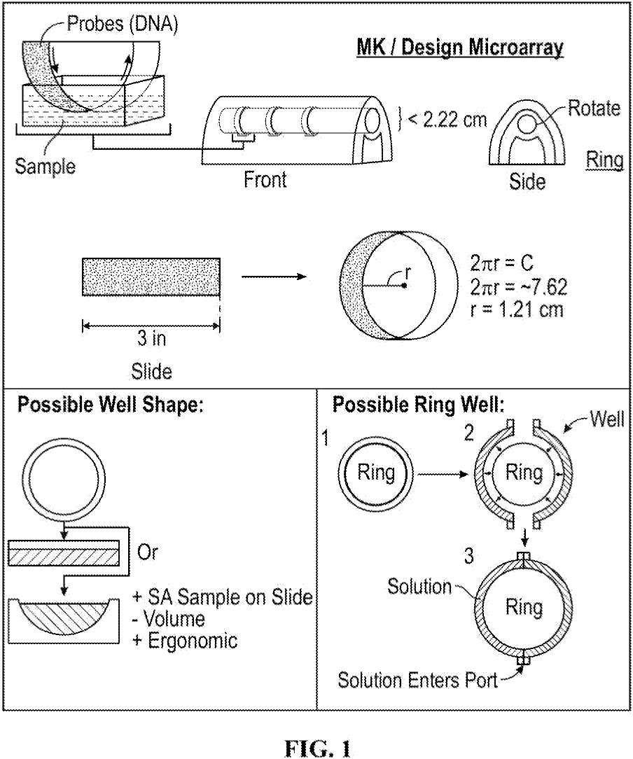

### DETAILED DESCRIPTION OF EXAMPLE EMBODIMENTS

Disclosed herein are a non-exhaustive series of methods and procedures designed to identify antibiotic resistance in clinical settings in a rapid and inexpensive manner. In one example, the procedure comprises several steps. In one example embodiment, a first step comprises a special method of bacterial cell isolation utilizing a centrifugation tube with either an inserted nanopore filter or a viscous gradient. In another embodiment, bacterial cells are lysed, and DNA is extracted using tubes prepared for a bead-beating process comprising beads and lysis buffer already measured and included inside. In this manner, a pre-treated 96 well plate set for multiplex PCR will admit to a standard, more rapid process of diagnosis. In another example embodiment, this process is used in conjunction with a method to distinguish amplicons using centrifugation and a protein that specifically binds to double or single-stranded DNA. In another embodiment, a microarray process is performed on a traditional flat glass or polymer slide using a machine that automates the process for easier for clinical use. In yet another embodiment, a novel microarray system admits to clinical antibiotic resistance characterization and pathogen identification. In a further embodiment, the microarray system utilizes an upright ring design for the microarray chip, thereby admitting to simpler automation and improved results.

In a first step of one example embodiment, a patient sample is first obtained. This can take many forms, such as a throat swab, stool sample, blood sample, etc. In a further embodiment, pathogen material is separated from human genetic material. This process admits to many advantages. The major advantage of selective filtration is that pathogen DNA will be at higher concentration, which in turn improves detection by any method. Furthermore, the human genome is typically several orders of magnitude larger than bacterial genomes. Small amounts of human cell contaminants can therefore result in an imbalance of genetic material in favor of the human genome. Removal of the human contaminants can also reduce processing time. Another advantage of removal is there is less likelihood of a false positive result due to a similar human sequence.

To achieve selective filtration, the patient specimen is initially processed by centrifugation in a special tube designed with a nanopore filter. In one example, the filter comprises a pore diameter range of between 5 and 50 micrometers. This allows for bacteria cells to pass through, while the larger eukaryotic cells of the patient remain behind the filter.

Another example method for selectively obtaining bacterial cells comprises centrifugation in a tube containing a viscous solution, for example, a sucrose solution. This process allows separation of components by mass. Bacterial cells have significantly less mass than the human cells of the patient. Thus, sample components will separate into distinct layers, of which the bacterial layer can be extracted for further processing.

After selective filtration of bacterial cells, the cells must be lysed so genetic material are extracted. In one embodiment, this process comprises preparing tubes consisting of glass or polymer beads and a lysis buffer optimized for bacteria. Bead-beating confers the advantage of extracting genetic material from cells with biofilm elements or a capsule. It also breaks genetic material into smaller pieces, which, depending on the size of beads and duration of beating, are then standardized to a desired range. This breaking of genetic material will aid hybridization efficiency in microarray procedures in lieu of restricting amplicon size via PCR.

After extraction of genetic material, the DNA is processed to confirm or disprove the presence and/or absence of particular genes conferring antibiotic resistance.

In some embodiments, PCR is then performed as a diagnostic procedure. For example, multiplex PCR has been used to identify antibiotic resistance and has proven effective. Its advantages include sensitivity, specificity, inexpensiveness, and rapidity. PCR also has several disadvantages, however. A principal disadvantage of this method is the complex technical knowledge required to perform PCR.

In order to make the procedure more simple and time effective in the clinic, either 96 or 128 well plates are coated with the proper primers, polymerase, nucleotides, and anything else required for the operation of a diagnostic PCR test already added to wells.

Other improvements are realized by omitting of an associated gel electrophoresis process. In order to increase the scope of diagnosis, each well contains a different set of primers. Conventional multiplex PCR identifies the primer or primers which were amplified by choosing primers with different amplicon lengths or fluorescent labels and then running the resultant through gel electrophoresis. This is not practical, however, for 96 wells with distinct primers in each.

Another example embodiment includes radioactive or fluorescent label primers in the pre-treated plate. After amplification, each of the 96 sets of different primers are added to a centrifuge tube along with a protein that binds selectively to double-stranded DNA. The protein will add a significant amount of weight to amplicons, which are then separated from primers that did not amplify any matter. The fluorescence or radioactivity of the primers incorporated into the amplicons are then determined and the results interpreted.

An alternative method used to identify antibiotic resistance genes using a PCR diagnostic test is a microarray system. In one example embodiment, a first step after extraction of genetic material comprises PCR used for a different purpose. In particular, PCR amplification is performed with general primers. This approach increases the amount of genetic material in certain samples in which normally only small amounts of bacteria are present.

However, in the majority of clinical samples with a problematic infection, PCR should not be necessary. This is due to a property of high-density microarrays, viz., that high-density microarrays typically comprise an overabundance of probes to increase sensitivity. In one embodiment, there are a great concentration of probes, which detect small amounts of complementary genetic material.

After PCR has been performed or omitted, respectively, DNA is labelled by a biotin or other molecular fluorescence or radioactive label. The DNA is then run through the microarray procedure. In this step, the advantages of automation are realized. For example, a device that automates the procedures necessary to run extracted microbial DNA from a clinic sample over a high-density spotted microarray chip is formed comprising a glass or a polymer material. The device is miniaturized and houses a central bay for insertion of a single microarray glass slide. Once inserted, the device is activated to perform a standardized procedure for diagnosis of antibiotic resistance. In one embodiment, this process comprises one or more of a hybridization, wash, and fluorescence scanning subroutines. In another embodiment, the device is connected via USB or another suitable means to a computer, where it is compatible with software for analysis and interpretation of results.

Such devices comprise a significant improvement over previously known microarray procedures utilizing manual use of materials, since such procedures are not practical enough to achieve prevalence in clinical use due to the expertise necessary to conduct microarray procedures as they stand today. A small device capable of automating the process from hybridization through data interpretation, however, is much more efficient and easily be employed at the clinical level.

Other advantages of miniature, multipurpose microarray systems comprise allowing for easier automation and a faster, cheaper, more sensitive and efficient process, in part because many more chips can be run at the same time. Also, the shape of the chips allows for a simpler method of automation of the hybridization process. This process takes advantage of the physical advantage of different geometric shapes. In known microarrays, a flat, rectangular piece of glass must have a solution oscillated over it for hours. The flat glass designed for probes often complements an agitation apparatus shaped like a basket. This configuration for agitating solutions across the microarray chip is bulky, however, which limits the number of probes that can occupy a machine at one time. By changing the probe shape from a flat plane to a ring, the same amount of space is utilized more efficiently. The result is an increased number of microarray chips that can simultaneously occupy a machine of similar size.

The presently disclosed microarray chip is manufactured by immobilizing probe sequences on a long, thin, flat polymer surface that is then curved and fused to form a ring structure. The ring structure affords many physical benefits. For example, the first part of the process is hybridization of genetic material to probes on the microarray surface. The ring microarray is advantageous in its annular shape. Current microarray hybridization machines are expensive because they must ensure that the hybridization buffer and genetic material is evenly spread across the flat surface and encounters each probe while simultaneously incubating. This process is complex, however, and must typically run for a long time or hybridization efficiences are detrimentally affected.

In contrast, a thin, ring-shaped microarray need only be rotated in a circular fashion to achieve the same purpose of contacting every probe, which is significantly easier to automate. In one embodiment, the hollow center of the ring is utilized to provide a heating source and uniformly incubate the array. Furthermore, the genetic material is more likely to be in close proximity to its complementary probe because the chip has much less width and the probe will therefore encounter sample genetic material every time the ring makes a full circle. This feature increases hybridization efficiency, thereby allowing for a more sensitive and rapid procedure.

The next process in a microarray procedure is to wash off non-complementary genetic material so that the chip can be scanned and read for fluorescence and then analyze the data. The ring, in an upright position, enables gravity to pull contents to the bottom, while genetic material hybridized to the immobilized probe sequences will migrate to the top. Thus, a light or even no wash will suffice if the reading system is located at the top of the ring because only hybridized material will make it to the top of the ring and be scanned for fluorescence.

Yet another advantage is that a protein with the capability to bind selectively to double-stranded DNA can be radioactively or fluorescently labeled and added in. Due again to gravity, this protein is only present at the top of the array if bound to genetic material that has hybridized to a probe. Thus, the entire procedure of hybridization, washing, and reading can be automated simply, with a slow circular motion of the ring and positioning of the heating system in the center of the ring and fluorescence scanner at the top of it.

In a further embodiment still, a protein is used to selectively bind to single stranded DNA. There is a class of proteins called single-stranded binding proteins that would be suitable for this purpose, though other suitable candidates could of course be employed with equal or perhaps even greater efficacy.

In yet another embodiment, the system comprises a software or other computer-based management package that uses data collection and data interpretation to draw trends, forecast changes in a community, as well as share data with government entities and/or companies to collaborate or act on said data. In one example embodiment, following the result of a positive test for an antibiotic resistant disease (or any disease for which monitoring is desired), the result of the test is uploaded to a central database. The test result is the only information sent, with no other patient info being uploaded. The gathered data is then used to predict and analyze the trends of disease and disease resistance in any given population to be used by government agencies (or a private entity) to help fight outbreaks of disease, in some cases before it is even recognized that such disease is present in the community. In a further embodiment, advanced machine learning, pattern detection, forecasting, etc., are used to enhance results.

The foregoing specification is provided for illustrative purposes only, and is not intended to describe all possible aspects of the present invention. Moreover, while the invention has been shown and described in detail with respect to several exemplary embodiments, those of ordinary skill in the relevant arts will appreciate that minor changes to the description, and various other modifications, omissions and additions may also be made without departing from either the spirit or scope thereof.

1. An integrated automated system for diagnosis of antibiotic resistance genes, wherein said system performs microarray hybridization, washes, and reading, and wherein said system further comprises a plurality of glass, probes and/or polymer microarray chips.

2. The system of claim 1, further comprising a plurality of ring or circle-shaped microarray chips, said chips further comprising a glass and/or a polymer.

3. The system of claim 1, further comprising a plurality of well plates, wherein said well plates are pre-treated with one or more of fluorescently labeled primers, polymerase, dNTPs or other chemicals or reagents used to multiplex PCR identification of antibiotic resistance genes.

4. The system of claim 3, wherein said well plates further comprise one or more of 96 or 128 sized well plates.

5. The system of claim 1, further comprising one or more centrifugation tubes comprising a nanopore filter fixed in place and/or a viscous solution for isolating smaller pathogens from larger host eukaryotic cells.

6. The system of claim 1, further comprising a software or other computer-based management package that uses data collection and data interpretation to draw trends, forecast changes in a community, as well as share data with government entities and/or companies to collaborate or act on said data.
